(19) United States
(12) Patent Application Publication
(10) Pub. No.: US 2015/0100111 A1
(43) Pub. Date: Apr. 9, 2015

(54) VISION RESTORATION ASSIST APPARATUS

(71) Applicant: NIDEK CO., LTD., Aichi (JP)

(72) Inventor: Masayuki SHINOMIYA, Aichi (JP)

(21) Appl. No.: 14/504,753

(22) Filed: Oct. 2, 2014

(30) Foreign Application Priority Data
Oct. 4, 2013 (JP) 2013-298702

(51) Int. Cl. A61N 1/375; A61N 1/36

(52) U.S. Cl. CPC A61N 1/375; A61N 1/36046; A61N 1/0543
USPC 607/115; 427/2.24

(57) ABSTRACT

A vision restoration assist apparatus includes an insulating case including a biocompatible body portion and a biocompatible cover portion that are joined to each other to form a storage space in the case, an electrode disposed outside the case, an electronic circuit disposed in the storage space of the case, and a joint layer disposed between joint surfaces of the body portion and the cover portion of the case and formed of a sintered bulk of a predetermined metal powder having an average particle size of 1 μm or less.

### CROSS-REFERENCE TO RELATED APPLICATION

This application claims priority from Japanese Patent Application No. 2013-298702 filed with the Japan Patent Office on Oct. 4, 2013, the entire content of which is hereby incorporated by reference.

### BACKGROUND

1. Technical Field

The present disclosure relates to a vision restoration assist apparatus including a device embedded in the body of a patient.

2. Related Art

In recent years, there has been research on a vision restoration assist apparatus for taking over some of the visual function that has been lost. For example, a known vision restoration assist apparatus includes an electronic circuit to which a number of electrodes are connected, the electronic circuit being embedded in the body of a patient such as the eye. The apparatus stimulates cells and the like of the patient associated with vision by outputting a current from the electronic circuit via the electrodes. In some of this type of apparatus, the electronic circuit is hermetically sealed in a case so as to protect the electronic circuit from the patient's bodily fluid. For example, the electronic circuit described in JP-A-2008-055000 is hermetically sealed between a mount and a cover portion that are joined to each other.

### SUMMARY

A vision restoration assist apparatus includes: an insulating case including a biocompatible body portion and a biocompatible cover portion, the body portion and the cover portion being joined to each other to form a storage space in the case; an electrode disposed outside the case; an electronic circuit disposed in the storage space of the case and configured to output a current via the electrode to electrically stimulate a vision-related cell or tissue of a patient; a connection terminal penetrating the case and electrically connecting the electronic circuit and the electrode; and a joint layer disposed between joint surfaces of the body portion and the cover portion of the case and formed of a sintered bulk of a predetermined metal powder having an average particle size of 1 μm or less.

### BRIEF DESCRIPTION OF DRAWINGS

FIG. 1 illustrates the outline of a vision restoration assist apparatus according to an embodiment of the present disclosure;

FIGS. 2A and 2B illustrate the configuration of an intracorporeal device;

FIG. 3 is a cross-sectional view of a stimulation control device; and

FIG. 4 is a schematic representation of the manner of attachment of the intracorporeal device with respect to the patient's eye.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The electronic circuit may be destroyed by heat upon exposure to high temperature. Thus, it is desirable that the hermetic sealing process to which the case is subjected can be performed at a low temperature such that no trouble would be caused in the electronic circuit, and is such a process that good airtightness can be obtained.

One object of an embodiment of the present disclosure is to provide a vision restoration assist apparatus including an electronic circuit configured to be embedded in the body of a patient that is hermetically sealed in a case in a preferable manner.

A vision restoration assist apparatus according to an embodiment of the present disclosure includes: an insulating case including a biocompatible body portion and a biocompatible cover portion, the body portion and the cover portion being joined to each other to form a storage space in the case; an electrode disposed outside the case; an electronic circuit disposed in the storage space of the case and configured to output a current via the electrode to electrically stimulate a vision-related cell or tissue of a patient; a connection terminal penetrating the case and electrically connecting the electronic circuit and the electrode; and a joint layer disposed between joint surfaces of the body portion and the cover portion of the case and formed of a sintered bulk of a predetermined metal powder having an average particle size of 1 μm or less.

A method of manufacturing a case included in a vision restoration assist apparatus according to an embodiment of the present disclosure includes: preparing a biocompatible cover portion and a biocompatible body portion of the case; coating one of a joint surface of the cover portion and a joint surface of the body portion with a predetermined metal powder having an average particle size of 1 μm or less; forming a sintered body by heating the metal powder; and heating and pressurizing the sintered body with the joint surface of the cover portion and the joint surface of the body portion being superposed on each other.

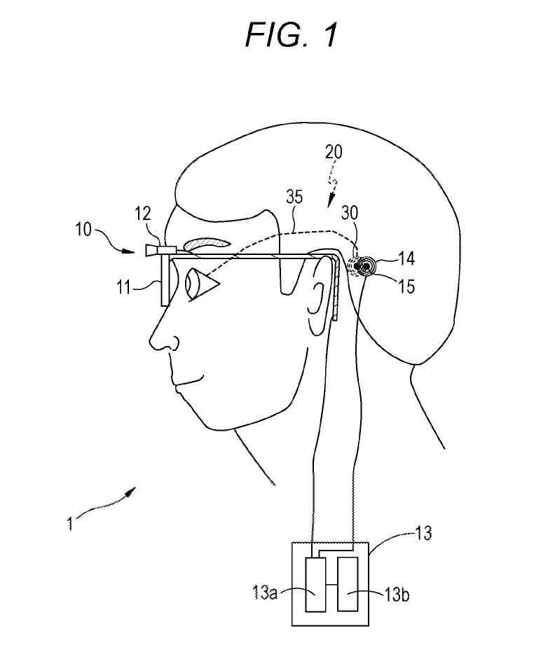

An exemplary embodiment of the present disclosure will be described below with reference to the drawings. First, a schematic configuration of a vision restoration assist apparatus 1 will be described with reference to FIG. 1.

The vision restoration assist apparatus 1 of the present embodiment promotes restoration of a patient's vision by electrically stimulating vision-related cells of the patient. As illustrated in FIG. 1, in the present embodiment, the vision restoration assist apparatus 1 includes an extracorporeal device 10 and an intracorporeal device 20. The extracorporeal device 10 acquires external information. The extracorporeal device 10 processes the acquired information and transmits a signal to the intracorporeal device 20. On the other hand, the intracorporeal device 20 of the present embodiment provides electrical stimulation to the retinal cells on the basis of the signal transmitted from the extracorporeal device 10.

In the present embodiment, the extracorporeal device 10 includes a visor 11, a photographing unit 12, an external unit 13, and a transmitter 14. The visor 11 has a spectacle shape and is worn in front of the patient's eye, as illustrated in FIG. 1. The photographing unit 12 is attached to the front surface of the visor 11, and photographs a subject forwardly of the patient. As the photographing unit 12, a CCD camera may be used, for example.

The external unit 13 includes a data modulation part 13a and a battery 13b. The battery 13b supplies electric power to the vision restoration assist apparatus 1 (the extracorporeal device 10 and the intracorporeal device 20). The data modulation part 13a includes a calculation controller such as a CPU. The data modulation part 13a generates a control signal for vision restoration by image-processing a subject image photographed by the photographing unit 12. The data modulation part 13a also superimposes the control signal on an electric power transmission signal by amplitude modulation or the like. As a result, the control signal and the electric power transmission signal are transmitted as an electromagnetic wave from a primary coil of the transmitter 14 to the intracorporeal device 20 (by wireless transmission, according to the present embodiment). At the center of the transmitter 14, a magnet 15 for affixing the position of the transmitter 14 with respect to a reception unit 30, which will be described later, is attached.

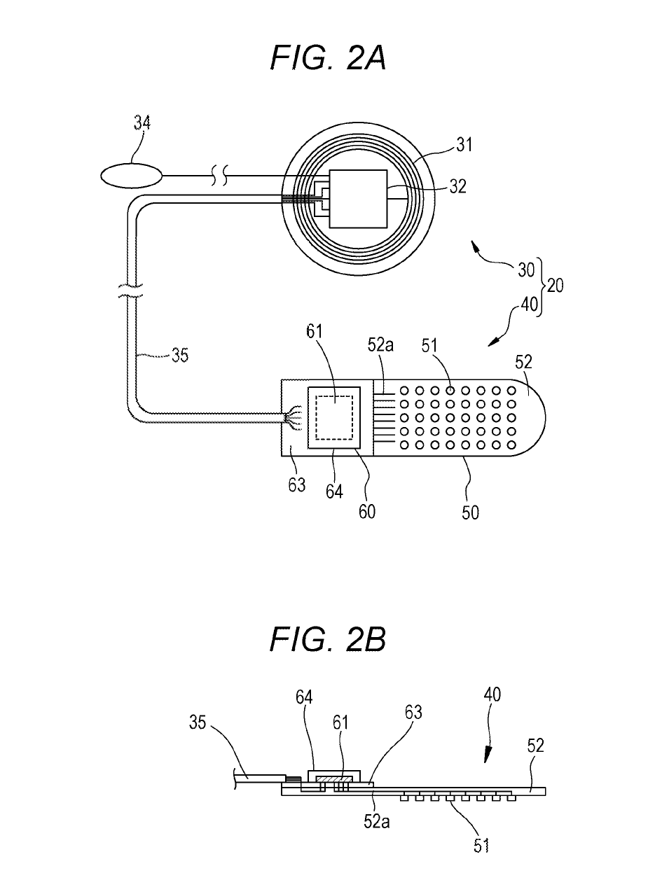

As illustrated in FIGS. 2A and 2B, the intracorporeal device 20 includes a reception unit 30 and a stimulation unit 40. The reception unit 30 receives the electromagnetic wave transmitted from the extracorporeal device 10. The reception unit 30 includes a receiver 31 and a controller (first internal signal output device) 32. In the present embodiment, the receiver 31 includes a secondary coil for receiving the electromagnetic wave from the extracorporeal device 10. The controller 32 extracts the control signal and electric power from the electromagnetic wave received by the receiver 31. The electric power extracted by the controller 32 is supplied to various parts of the intracorporeal device 20. The reception unit 30 is also provided with a magnet (not illustrated) for affixing the position of the transmitter 14.

As illustrated in FIG. 2A, a feedback electrode 34 is attached to the reception unit 30. While the details will be described later, the feedback electrode 34 is attached with the patient's eye (retina) disposed between the electrode and the stimulation unit 40. A voltage for guiding the current output from the stimulation unit 40 is applied to the feedback electrode 34.

As illustrated in FIG. 2A, the reception unit 30 and the stimulation unit 40 are connected by a lead wire 35. The controller 32 supplies electric power and also outputs a control signal to the stimulation unit 40 via the lead wire 35.

In the present embodiment, the stimulation unit 40 electrically stimulates the retinal cells. The stimulation unit 40 includes an electrode portion 50 and a stimulation controller (second internal signal output device) 60. In the present embodiment, the stimulation unit 40 is installed in the eyeball. In the present embodiment, particularly, a part of the stimulation unit 40 is installed between layers (or in the layers) of a layered eye tissue. For example, a part of the stimulation unit 40 is installed between the sclera and the choroid.

The electrode portion 50 serves as a multielectrode array including a substrate 52 and a plurality of electrodes 51 formed on the substrate 52. The electrodes 51 are arranged at equal intervals in a lattice shape, or at equal intervals in a staggered manner. The number of the electrodes 51 may be determined depending on the resolution at the time of vision restoration. In the present embodiment, an example will be described in which 54 electrodes 51 are formed on the substrate 52. Incidentally, this is merely an example and more or less electrodes 51 may be provided on the substrate 52. The electrodes 51 are formed of a biocompatible conductor. For example, the electrodes 51 may be formed of a precious metal such as gold or platinum.

The substrate 52 is an insulator and formed in plate shape. Preferably, the substrate 52 is formed of a biocompatible material. Preferably, the substrate 52 has a shape conforming to the eyeball shape. For example, the substrate 52 may be formed to be bendable with a required thickness. In the substrate 52, a plurality of lead wires 52a is provided. Via the lead wires 52a, the respective electrodes 51 are electrically connected to the stimulation controller 60.

As illustrated in FIGS. 2A and 2B, the stimulation controller 60 of the present embodiment includes an electronic circuit 61. In the present embodiment, the electronic circuit 61 includes a ceramic substrate, and a plurality of electronic components mounted on the ceramic substrate, such as a custom IC, capacitors, and diodes. In the present embodiment, the electronic circuit 61 is a demultiplexer. The electronic circuit 61 receives the control signal from the controller 32 via the lead wire 35 which will be described later. Based on the control signal, the electronic circuit 61 sorts a current (a bipolar pulse, according to the present embodiment) out to the respective electrodes 51.

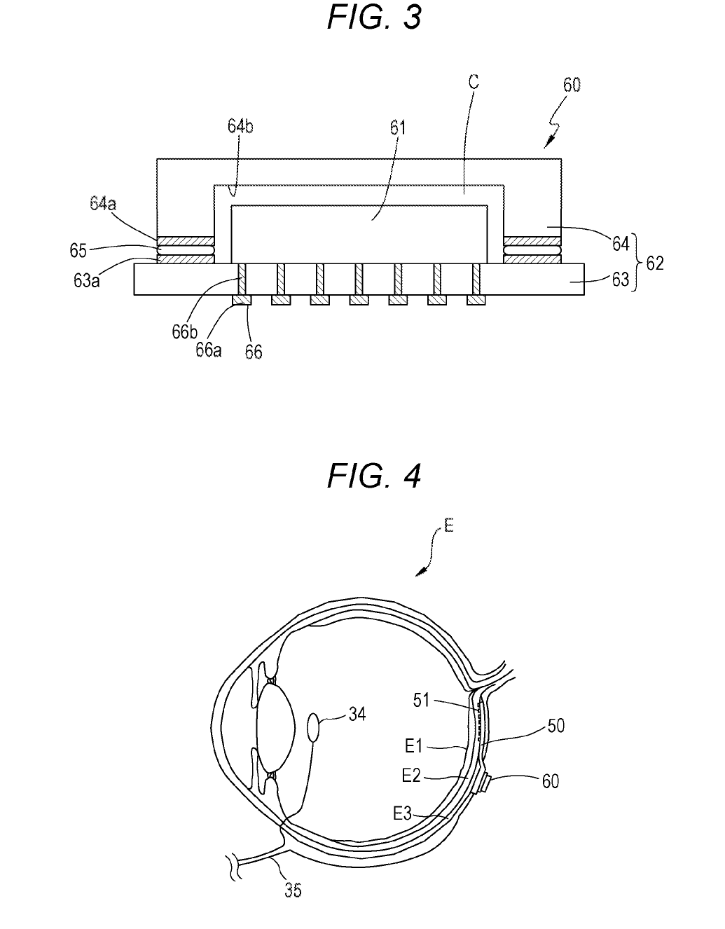

The stimulation controller 60 also includes a case 62 that seals the electronic circuit 61. With reference to FIG. 3, the detailed configuration of the stimulation controller 60 will be described. As illustrated in FIG. 3, the stimulation controller 60 includes a joint layer 65 and a plurality of connection terminals 66, in addition to the electronic circuit 61 and the case 62.

In the present embodiment, the case 62 includes a mount (body portion) 63 and a cover portion 64. A joint surface 63a of the mount 63 and a joint surface 64a of the cover portion 64 are joined, forming a storage space C for storing the electronic circuit 61. The mount 63 and the cover portion 64 have their own biocompatibilities.

In the present embodiment, the electronic circuit 61 is attached on the mount 63. In the present embodiment, for the mount 63, a flat ceramic plate is used. However, the material of the mount 63 is not limited to ceramic, and may include various biocompatible insulators. Given an extended period of time in which the stimulation controller 60 may be installed in the body, a material with little degradation in the body over time may be used for the mount 63. This condition may be met by, for example, glass and sapphire.

In order to decrease irregularities on the ceramic surface, in the present embodiment, the joint surface 63a of the mount 63 is ground. The joint surface 63a of the present embodiment is ground by a lapping process, and then ground by a polishing process. When the grinding is performed up to the polishing process, the joint surface 63a may be smoothened in a more preferable manner. Depending on the relationship between the state of the joint surface 63a and the accuracy required for hermetically sealing the electronic circuit 61, the polishing process may not be performed.

In the present embodiment, the joint surface 63a of the mount 63 is subjected to a sintering process (sintering) after the grinding. The sintering process herein refers to a thermal process whereby the mount 63 is heated so as to cause a glassy material of the mount 63 to be transported to the surface of the joint surface 63a. The sintering process implemented on the mount 63 enables fine depressions (voids) in the joint surface 63a that cannot be removed by grinding alone to be filled with the glassy material. As a result, the joint surface 63a can be made smoother. Through the sintering process, the mount 63 is heated to approximately 1000° C., for example. Thus, in the present embodiment, the grinding and the sintering processes for the joint surface 63a are performed before the electronic circuit 61 is attached on the mount 63.

On the joint surface 63a of the mount 63, a metal thin film (a metal layer, which is a gold (Au) thin film according to the present embodiment) is formed by a metallization process. As will be described later, the joint layer 65 of the present embodiment is formed of a metal. By thus locating the metal thin film between the joint surface 63a and the joint layer 65, the joint strength between the non-metal installation base 63 and the metal joint layer 65 can be ensured, while increasing the airtightness at their interface. In the present embodiment, the metal thin film is formed on the sintered mount 63 by a known metallization process such as vacuum deposition, sputtering, or active metal process.

As illustrated in FIG. 3, the mount 63 is provided with a plurality of connection terminals 66. The plurality of connection terminals 66 penetrate the mount from the surface to which the electronic circuit 61 is attached to the opposite surface. Some of the connection terminals 66 are each independently connected to the electrodes 51 via the lead wires 52a in the substrate 52. The remaining connection terminals 66 are connected to the controller 32 via the lead wire 35. By the connection terminals 6, the electronic circuit 61 is connected to an extracorporeal device while being hermetically sealed in the case 62.

In the present embodiment, the connection terminals 66 are formed of platinum (Pt). The connection terminals 66 may be formed of a biocompatible conductor other than platinum (such as a precious metal, including gold). However, it is particularly preferable to form the connection terminals 66 from platinum, which is not readily subjected to electrolysis in the body. The connection terminals 66 include a conductive via portion 66b penetrating the mount 63, and a terminal portion 66a exposed on the outside of the case 62. Of these potions, only the terminal portion 66a that may come into contact with the living body may be formed of platinum.

In the present embodiment, the cover portion 64 is formed with a recess 64b for storing the electronic circuit 61. The cover portion 64 is joined to the mount 63, covering the electronic circuit 61. Preferably, the cover portion 64 is formed of a biocompatible material that exhibits little degradation in the body over time, such as glass, sapphire, or certain ceramics. For the cover portion 64 of the present embodiment, glass is used.

On the joint surface 64a of the cover portion 64, there is also formed a metal thin film similar to the one on the joint surface 63a of the mount 63, so as to ensure join strength and airtightness between the joint surface 64a and the metal joint layer 65.

The joint layer 65 will be described. The joint layer 65 is formed by the process of joining the mount 63 and the cover portion 64 to each other. The joining process includes locating a predetermined metal powder (metal powder sintered body) having an average particle size of 1 μm or less between the joint surface 63a of the mount 63 and the joint surface 64a of the cover portion 64, and heating the metal powder.

The metal powder is applied to at least one of the joint surface 63a of the mount 63 and the joint surface 64a of the cover portion 64 in advance. The application of the metal powder may include applying a mixture (metal paste) of the metal powder and an organic solvent and the like to at least one of the joint surfaces 63a and 64a. For the metal powder applied to the joint surface 63a and/or 64a, a biocompatible metal may be used. In the present embodiment, a gold (Au) metal powder is used. As the metal powder applied to the joint surface 63a and/or 64a, a metal powder with an average particle size of 1 μm or less may be used. In the present embodiment, a gold metal powder with an average particle size of 0.3 μm or more and 0.5 μm or less is used. The average particle size in the present embodiment may be determined on the basis of a metal particle granularity distribution obtained by a photocentrifuge method. As the average particle size, the particle size at 50% of an integrated value of the granularity distribution may be used, for example.

The predetermined metal as the material of the metal powder is not necessarily limited to gold. The predetermined metal may include any biocompatible metal such that, when a sintered body is formed from the powder with the average particle size of 1 μm or less, sintering densification (bulk formation) can proceed when heated at a temperature of not higher than the melting point of a bulk body of the metal (such as 300° or lower) under pressurization. As the predetermined metal, a precious metal such as gold, silver, platinum, or palladium may be used while adjusting the particle size of the metal powder as needed. The metal powder may be a mixture powder of two or more kinds of metal satisfying the above condition.

In the present embodiment, the joint surface 64a of the cover portion 64 is coated with the metal powder. Thereafter, the cover portion 64 is heated at a predetermined temperature for a certain period of time, using an electric furnace and the like. This causes the metal powder to melt at mutually contacting portions and become bonded to an appropriate degree. As a result, a sintered body of the metal powder is formed. Preferably, the heating temperature at the time of sintering is 80° C. to 300° C. If the heating temperature is lower than 80° C., the metal particles do not readily form point contact, making it difficult to obtain a proper sintered body. On the other hand, if the heating temperature exceeds 300° C., the metal particles may become bonded to each other too strongly, making it difficult for the cover portion 64 to be joined to the mount 63 in the subsequent process. When the metal paste is applied to the cover portion 64, a step of removing residual organic solvent by drying the cover portion 64 may be performed after the sintered body is formed.

Next, the joint surface 63a of the mount 63 with the electronic circuit 61 mounted thereon is superposed with the joint surface 64a of the cover portion 64. Thereafter, the sintered body is pressurized while being heated. The metal powder constituting the sintered body becomes bonded to each other while being plastically deformed. Thus, the joint layer 65 joining the mount 63 and the cover portion 64 is formed. As a result, the electronic circuit 61 is hermetically sealed in the storage space C of the case 62. A favorable joint layer 65 may be readily obtained if the sintered body is heated at a temperature on the order of 80° C. to 300° C. This is because, if the temperature is lower than 80° C., the joint is not readily obtained, and if the temperature is 300° C. or more, the joint layer 65 may be readily subjected to the influence of thermal strain upon cooling. In the present embodiment, densification of the sintered body may be caused to proceed in a preferable manner by applying a pressure of 100 Mpa or more at the time of heating. The joint layer 65 may be obtained more preferably by increasing the pressure applied to the sintered body. However, if the pressure applied to the sintered body is higher than about 200 Mpa, the effect of pressurization becomes substantially constant. The joint layer 65 obtained by applying heat and pressure to the sintered body is a bulk body of high-purity metal. Thus, the joint layer 65 would not become melted again unless heated at a high temperature (around the melting point of the metal). Accordingly, when the mount 63 and the cover portion 64 are joined by the above method, airtightness in the case 62 can be easily maintained. In this manner, the electronic circuit 61 is stored in the highly airtight case 62. It is noted that, in a helium leakage test conducted by the present discloser, a sample having the same configuration as the case 62 of the present embodiment sometimes showed high airtight performance of 1.0×10⁻¹³ Pa·m³/S or less.

Preferably, the heating temperature during the joining is set sufficiently low relative to the heatproof temperature of the electronic circuit 61. This is because the lower the heating temperature during the joining, the less likely it is that the electronic circuit 61 will be destroyed by the influence of heat during the joining.

With reference to FIG. 4, an example of the manner of attachment of the intracorporeal device (intraocular device) 20 to the patient's eye will be described. As illustrated in FIG. 4, the intracorporeal device 20 is configured to stimulate retinal cells E1 using the electrodes 51 installed on the choroid E2. In this case, the stimulation unit 40 is installed in the patient's eye by inserting the front-end portion of the substrate 52 with the electrodes 51 into a pocket created by cutting open a part of the sclera E3. The feedback electrode 34 is placed close to the anterior segment. Thus, the retina E1 is positioned between the electrodes 51 and the feedback electrode 34. The stimulation controller 60 outputs a current via the electrodes 51 while a voltage is being applied to the feedback electrode 34, whereby the stimulation current can efficiently penetrate the retina, and electrical stimulation is preferably applied to the retina.

As described above, in the vision restoration assist apparatus 1 according to the present embodiment, the electronic circuit 61 is hermetically sealed in the storage space C of the case 62 that is formed when the mount 63 and the cover portion 64 are joined. The mount 63 and the cover portion 64 are joined by the joint layer 65 disposed between the joint surface 63a and the joint surface 64a. The joint layer 65 is formed by sintering a predetermined metal powder (which is gold powder in the present embodiment) having an average particle size of 1 μm or less into a bulk. As described above, the joint layer 65 may be formed at a temperature sufficiently lower than the melting point temperature of the metal bulk body. Thus, destruction of the electronic circuit 61 due to heat at the time of hermetic sealing, for example, can be suppressed. The larger the amount of heat that is transmitted to the electronic circuit 61 from the case 62 during the joining of the case 62, the greater the margin may be that is set with respect to the size of the case 62 hermetically sealing the electronic circuit 61. As described above, in the vision restoration assist apparatus 1 according to the present embodiment, the joining of the case 62 may be performed at low temperature. Thus, the electronic circuit 61 can be easily hermetically sealed in a preferable manner even when the size of the case 62 is small, for example.

In the vision restoration assist apparatus 1, particularly, the electronic circuit 61 has high airtightness such that water (water vapor) does not easily enter the case. Thus, the electronic circuit 61 is not easily degraded by moisture. Accordingly, the electronic circuit 61 can operate stably for a long period with the electronic circuit 61 (stimulation unit 40) embedded in the body.

In the vision restoration assist apparatus 1 according to the present embodiment, the joint surface 63a of the mount 63 made of ceramic material is treated by sintering process. The sintering process smoothens the surface of the joint surface 63a. Thus, during the joining process using metal powder (metal powder sintered body), a gap is not readily formed between the joint layer 65 and the joint surface 63a. Accordingly, in the vision restoration assist apparatus 1, the electronic circuit 61 can be easily hermetically sealed in a preferable manner.

Further, in the present embodiment, the sintering process is performed on the joint surface 63a that has been subjected to polishing process in advance. As a result, the joint layer 65 is joined to the joint surface 63a that is extremely smooth, so that the electronic circuit 61 can be hermetically sealed in the vision restoration assist apparatus 1 in an even more preferable manner.

In the foregoing, the present disclosure has been described with reference to an embodiment. However, it should be apparent that the present disclosure is not limited to the embodiment and that various modifications can be made.

For example, in the embodiment, the mount 63 of the case 62 and the cover portion 64 are formed of mutually different materials. However, the mount 63 and the cover portion 64 may be formed of the same material. The mount 63 and the cover portion 64 are both thermally expanded by the heat during the joining process. At this time, the greater the difference in thermal expansion coefficient between the mount 63 and the cover portion 64 in the range of from normal temperature to the temperature at the time of the joining process, the more readily strain is caused in the joint layer 65 upon cooling, making it difficult to seal the electronic circuit 61 in a preferable manner. On the other hand, when the mount 63 and the cover portion 64 are formed of the same material, approximately the same degree of expansion is caused in both, making it difficult for strain to be caused in the joint layer 65, enabling the electronic circuit 61 to be readily hermetically sealed in a preferable manner. Further, even when the mount 63 and the cover portion 64 are formed of mutually different materials, the strain due to thermal expansion at the time of joining is not readily caused in the joint layer 65, and the electronic circuit 61 can be hermetically sealed in a preferable manner, if the thermal expansion coefficients of both materials are close in the range of from normal temperature to the temperature during the joining process.

In the embodiment, the electrodes 51 are installed in the eyeball, and the retinal cells are stimulated. However, this is merely an example, and an embodiment of the present disclosure may be configured such that restoration of the patient's vision is promoted by stimulating the cells or tissue associated with the patient's vision. For example, the electrodes may be installed in the optic nerves or the cerebral cortex.

All of or a part of the configuration of the reception unit 30 may be included in the stimulation controller 60 of the stimulation unit 40. In this case, the stimulation controller 60 may be disposed away from the patient's eye (i.e., from the electrode portion 50).

In the embodiment, for the metal thin film formed on the mount 63 by metallization process, the same material as for the joint layer 65 (gold in the embodiment) is used. Alternatively, they may be formed of mutually different materials.

The vision restoration assist apparatus according to an embodiment of the present disclosure may include the following first to fourth vision restoration assist apparatuses.

A first vision restoration assist apparatus includes an insulating case having a body portion and a cover portion, the body portion and the cover portion being joined, forming a storage space; an electrode disposed outside the case; an electronic circuit disposed in the storage space of the case and configured to output a current via the electrode to electrically stimulate a vision-related cell or tissue of a patient; and a connection terminal penetrating the case and electrically connecting the electronic circuit and the electrode. The body portion and the cover portion of the case are of biocompatible and are joined to each other via a joint layer formed of a sintered bulk of metal powder of a predetermined metal disposed between joint surfaces of the body portion and the cover portion and having an average particle size of 1 μm or less, with the electronic circuit hermetically sealed in the storage space.

A second vision restoration assist apparatus is the first vision restoration assist apparatus wherein at least one of the body portion and the cover portion is formed of ceramic, with the joint surface thereof being treated by a sintering process.

A third vision restoration assist apparatus is the second vision restoration assist apparatus wherein the sintering process is provided to the joint surface that is polished in advance.

A fourth vision restoration assist apparatus is the first vision restoration assist apparatus wherein the predetermined metal includes at least one of gold, silver, platinum, and palladium.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A vision restoration assist apparatus, comprising:
an insulating case including a biocompatible body portion and a biocompatible cover portion, the body portion and the cover portion being joined to each other to form a storage space in the case;
an electrode disposed outside the case;
an electronic circuit disposed in the storage space of the case and configured to output a current via the electrode to electrically stimulate a vision-related cell or tissue of a patient;
a connection terminal penetrating the case and electrically connecting the electronic circuit and the electrode; and
a joint layer disposed between joint surfaces of the body portion and the cover portion of the case and formed of a sintered bulk of a predetermined metal powder having an average particle size of 1 μm or less.

2. The vision restoration assist apparatus according to claim 1, wherein at least one of the body portion and the cover portion is formed of ceramic and includes a joint surface that has been subjected to a sintering process.

3. The vision restoration assist apparatus according to claim 2, wherein the joint surface that has been subjected to the sintering process is a joint surface that is ground in advance.

4. The vision restoration assist apparatus according to claim 1, wherein the predetermined metal includes at least one of gold, silver, platinum, and palladium.

5. A method of manufacturing a case included in a vision restoration assist apparatus, comprising:
preparing a biocompatible cover portion and a biocompatible body portion of the case;
coating one of a joint surface of the cover portion and a joint surface of the body portion with a predetermined metal powder having an average particle size of 1 μm or less;
forming a sintered body by heating the metal powder; and
heating and pressurizing the sintered body with the joint surface of the cover portion and the joint surface of the body portion being superposed on each other.

6. The method according to claim 5, further comprising:
forming at least one of the joint surface of the cover portion and the joint surface of the body portion with ceramic; and
subjecting the joint surface formed of ceramic to a sintering process.

7. The method according to claim 6, further comprising:
grinding the joint surface formed of ceramic prior to the sintering process.

8. The method according to claim 5, wherein the predetermined metal includes at least one of gold, silver, platinum, and palladium.
